(19) United States
(12) Patent Application Publication
(10) Pub. No.: US 2024/0068041 A1
(43) Pub. Date: Feb. 29, 2024

(54) FREE DNA-BASED DISEASE PREDICTION MODEL AND CONSTRUCTION METHOD THEREFOR AND APPLICATION THEREOF

(71) Applicant: BGI SHENZHEN, Shenzhen (CN)

(72) Inventors: Jia Ju, Shenzhen (CN); Yong Bai, Shenzhen (CN); Ruoyan Chen, Shenzhen (CN); Xin Jin, Shenzhen (CN)

(21) Appl. No.: 18/261,282

(22) PCT Filed: Jan. 14, 2021
(86) PCT No.: PCT/CN2021/071822

§ 371 Date: Jul. 13, 2023

(51) Int. Cl. C12Q 1/6886; G16B 25/10; G16B 30/10

(52) U.S. Cl. CPC C12Q 1/6886; G16B 25/10; G16B 30/10

(57) ABSTRACT

Described is a free DNA-based disease prediction model and a construction method therefor and an application thereof. The construction method includes the steps of: 1) obtaining sequencing data of free DNA samples of diseased individuals and control individuals, the number of the diseased individuals and the number of the control individuals being both multiple; 2) selecting, according to the coverage of the sequencing data of the free DNA samples of the diseased individuals and the control individuals on a genome, a gene set having a difference in the coverage of a transcription initiation site region between the diseased individuals and the control individuals; and 3) for genes in the gene set, using the coverage of the sequencing data on the gene transcription initiation site region as an input prediction model for training so as to establishing a disease prediction model.

### FIELD OF THE INVENTION

The present invention belongs to the field of biotechnology, and more specifically, relates to a method for disease prediction by using cell-free DNA.

### BACKGROUND OF THE INVENTION

Tumor prediction is an important problem in the prior art, and many methods that can be applied to tumor prediction at present. Tumor prediction is conducted based on serological tumor markers, and many serum proteins such as CA125, CA19-9, CEA, HGF and the like, play a certain role in the diagnosis and detection of tumors [1, 2]. CT, nuclear magnetic resonance and other imaging means are used for tumor prediction. Gene prediction may base on the next-generation sequencing technology as follows. A) Tumor prediction may base on genomic variation at SNV level. Recent studies on cfDNA show that tumor-specific mutation studies can be used for early screening of tumors, in which tumor-specific somatic mutation can be detected by targeted sequencing with high depth or multiplex PCR, etc. [3, 4]. B) Tumor prediction may base on CNV. Variation at chromosome level or copy number variation can be detected by cfDNA whole genome sequencing [5-7]. C) Tumor prediction may base on chromosomal methylation. Recent studies show that methylation biomarkers can be used for tumor prediction [8, 9]. D) Tumor prediction may base on the specific nucleosome-associated blotting of the cfDNA fragment of tumor. CfDNA sequencing can reflect the length of the encapsulated nucleosome cfDNA fragment. The study by Jiang P et al. [7] pointed out that the cfDNA fragments of patients with liver cancer would be partially shorter than those of normal individuals in the detection of tumor fragments in the cfDNA of patients with liver cancer. Cristiano S et al. take the proportion of short fragments of cfDNA in each interval of the whole genome as a feature, which can be used to predict tumors and identify tissue types thereof. The positions of nucleosomes and the position of the end of cfDNA fragments on genome [12, 13] show a certain correlation with the tumor and its tissue source.

These above techniques are usually used in combination in existing tumor detection products and published tumor prediction research results. For example, LUNAR-2 (https://guardanthealth.com/solutions/#lunar-2) of Guardant Health is a combination of the above techniques of A), C), and D), and can reach a higher sensitivity in colorectal cancer detection. However, the specific method is unknown. Signature (https://www.natera.com/signatera), a postoperative tumor detection product of Natera company, based on the above A), selects 16 specific SNV loci, which can reach an ultrahigh sensitivity in the recurrence detection of colorectal cancer and lung cancer [14, 15]. Joshua D.cohen's team published a study in Science in 2018: CancerSEEK, a tumor detection method based on serum markers and SNV, shows a specificity of up to 99% and a sensitivity of 69% to 98% depending on cancer type when used in 1005 patients with 8 different types of tumors including lung cancer, liver cancer, colorectal cancer, etc [16].

There are some main shortcomings in tumor prediction in the prior art. For example, serological tumor markers usually exist simultaneously in the serum of normal individuals, which leads to lower precision and specificity in detection, so it is difficult to be applied in the early screening of tumors. There is a higher risk of false positive and false negative in the early screening of tumors when using CT, nuclear magnetic resonance and other imaging means for detection, and it is difficult to realize early screening of tumors. Gene detection based on next-generation sequencing technology may have the following problems. For detection based on genomic variation at SNV level, the specific variation cannot be detectable in all patients, and it is difficult to achieve large-scale popularization due to the high experimental cost. For detection based on CNV, only a small number of individuals have this type of variation. For detection based on genomic methylation, it is difficult to achieve large-scale application and popularization due to the higher cost. For detection based on the specific nucleosome-associated blotting of the cfDNA fragments of the tumor, it usually requires higher sequencing depth, and it is only in the stage of scientific research, and is difficult to be applied in clinical routine detection. In summary, there is no effective method for predicting early tumors in the prior art.

### REFERENCES

1. Patz, E. F., Jr., et al., Panel of serum biomarkers for the diagnosis of lung cancer. J Clin Oncol, 2007. 25(35): p. 5578-83.
2. Liotta, L. A. and E. F. Petricoin, 3rd, The promise of proteomics. Clin Adv Hematol Oncol, 2003. 1(8): p. 460-2.
3. Phallen, J., et al., Direct detection of early-stage cancers using circulating tumor DNA. Sci

Transl Med, 2017. 9(403).
4. Bettegowda, C., et al., Detection of circulating tumor DNA in early- and late-stage human malignancies. Sci Transl Med, 2014. 6(224): p. 224ra24.
5. Leary, R. J., et al., Detection of chromosomal alterations in the circulation of cancer patients with whole-genome sequencing. Sci Transl Med, 2012. 4(162): p. 162ra154.
6. Chan, K. C., et al., Noninvasive detection of cancer-associated genome-wide hypomethylation and copy number aberrations by plasma DNA bisulfite sequencing. Proc Natl Acad Sci USA, 2013. 110(47): p. 18761-8.
7. Jiang, P., et al., Lengthening and shortening of plasma DNA in hepatocellular carcinoma patients. Proc Natl Acad Sci USA, 2015. 112(11): p. E1317-25.
8. Hao, X., et al., DNA methylation markers for diagnosis and prognosis of common cancers. Proc Natl Acad Sci USA, 2017. 114(28): p. 7414-7419.
9. Guo, S., et al., Identification of methylation haplotype blocks aids in deconvolution of heterogeneous tissue samples and tumor tissue-of-origin mapping from plasma DNA. Nat Genet, 2017. 49(4): p. 635-642.
10. Cristiano, S., et al., Genome-wide cell-free DNA fragmentation in patients with cancer. Nature, 2019. 570(7761): p. 385-389.
11. Snyder, M. W., et al., Cell-free DNA Comprises an In Vivo Nucleosome Footprint that Informs Its Tissues-Of-Origin. Cell, 2016. 164(1-2): p. 57-68.
12. Jiang, P., et al., Preferred end coordinates and somatic variants as signatures of circulating tumor DNA associated with hepatocellular carcinoma. Proc Natl Acad Sci USA, 2018. 115(46): p. E10925-E10933.
13. Sun, K., et al., Orientation-aware plasma cell-free DNA fragmentation analysis in open chromatin regions informs tissue of origin. Genome Res, 2019. 29(3): p. 418-427.
14. Abbosh, C., et al., Phylogenetic ctDNA analysis depicts early-stage lung cancer evolution. Nature, 2017. 545(7655): p. 446-451.
15. Reinert, T., et al., Analysis of Plasma Cell-Free DNA by Ultradeep Sequencing in Patients With Stages I to III Colorectal Cancer. JAMA Oncol, 2019.
16. Cohen, J. D., et al., Detection and localization of surgically resectable cancers with a multi-analyte blood test. Science, 2018. 359(6378): p. 926-930.

### SUMMARY OF THE INVENTION

In view of the current situation that there is no effective disease diagnosis method in clinical practice, the present invention attempts to provide a disease prediction model with a relatively high accuracy and its construction method and application.

Therefore, in a first aspect, the present invention provides a method for constructing a cell-free DNA-based disease prediction model, comprising:
1) obtaining sequencing data of cell-free DNA samples of a plurality of diseased individuals and a plurality of control individuals;
2) selecting a gene set having differences in the coverage at the transcription start site regions between the diseased individuals and the control individuals according to the coverage of the sequencing data of the cell-free DNA samples of the diseased individuals and the control individuals on the genome; and
3) for the genes in the gene set, training a prediction model by inputting the coverage of the sequencing data at the gene transcription start site regions to construct a disease prediction model.

In one embodiment, the disease is cancer, and preferably, the cancer is lung cancer, liver cancer or colorectal cancer.

In one embodiment, the disease prediction includes early screening of tumors or detection of tumor recurrence.

In one embodiment, in 1), the cell-free DNA samples are derived from body fluids, such as blood.

In one embodiment, in 2), the coverage of the cell-free DNA on the genome is determined by the relative coverage.

In one embodiment, in 2), the transcription start site region refers to a region of 100 bp, 400 bp, 600 bp, or 1 kb upstream and downstream of the transcription start site.

In one embodiment, in 2), the genes having differences in the coverage at the transcription start site regions between the diseased individuals and the control individuals are sorted, and the genes with large value are selected.

In one embodiment, in 2), the gene set comprises 10-50 genes.

In one embodiment, in 3), the prediction model is a Logistic Regression model or a Random Forest model.

In a second aspect, the present invention provides a disease prediction model constructed according to the method of the first aspect of the present invention.

In a third aspect, the present invention provides a cell-free DNA-based disease prediction method, which uses the disease prediction model constructed according to the method of the first aspect of the present invention, comprising:
1) for the cell-free DNA sample of an individual to be tested, obtaining sequencing data of the gene set determined in constructing the disease prediction model;
2) for the genes in the gene set, obtaining the coverage of the sequencing data at the transcription start site regions; and
3) inputting the coverage at the transcription start site regions into the disease prediction model to predict whether the individual to be tested suffers from the disease.

In a fourth aspect, the present invention provides a cell-free DNA-based disease prediction system, comprising:
a sequence acquisition unit, configured to obtain sequencing data of cell-free DNA samples of a plurality of diseased individuals, a plurality of control individuals and an individual to be tested;
a gene set selection unit, configured to select a gene set having differences in the coverage at the transcription start site regions between the diseased individuals and the control individuals according to the coverage of the sequencing data of the cell-free DNA samples of the diseased individuals and the control individuals on the genome;
a model constructing unit, configured to, for the genes in the gene set, train a prediction model by inputting the coverage of the sequencing data of the diseased individuals and the control individuals at the gene transcription start site regions to construct a disease prediction model; and
a prediction unit, configured to, for the genes in the gene set, input the coverage of the sequencing data of the individual to be tested at the gene transcription start site regions into the disease prediction model to predict whether the individual to be tested suffers from the disease.

In one embodiment, the disease is cancer, and preferably, the cancer is lung cancer, liver cancer or colorectal cancer.

In one embodiment, the disease prediction includes early screening of tumors or detection of tumor recurrence.

In one embodiment, in the sequence acquisition unit, the cell-free DNA samples are derived from body fluids, such as blood.

In one embodiment, in the gene set selection unit, the coverage of the cell-free DNA on the genome is determined by the relative coverage.

In one embodiment, in the gene set selection unit, the transcription start site region refers to a region of 100 bp, 400 bp, 600 bp, or 1 kb upstream and downstream of the transcription start site.

In one embodiment, in the gene set selection unit, the genes having differences in the coverage at the transcription start site regions between the diseased individuals and the control individuals are sorted, and the genes with large value are selected.

In one embodiment, in the gene set selection unit, the gene set comprises 10-50 genes.

In one embodiment, in the model constructing unit, the prediction model is a Logistic Regression model or a Random Forest model.

The present invention realizes rapid, efficient and low-cost early prediction of diseases such as lung cancer by using only the sequencing depth distribution information of cfDNA in one sampling without using any other assistant means and additional data.

### BRIEF DESCRIPTION OF THE DRAWINGS

FIG. 1 is the ROC curve of the test set of lung cancer with an area under the curve (AUC) of 0.75.

FIG. 2 is the ROC curve of the test set of liver cancer with an area under the curve (AUC) of 1.00.

### DETAILED DESCRIPTION OF THE EMBODIMENTS

Peripheral blood of tumor patients contains circulating tumor DNA (ctDNA) derived from tumor. CtDNA only accounts for a small part of all circulating cell-free DNA (cfDNA) in the peripheral blood. The present invention utilizes the changes of coverage depth of sequencing reads of cfDNA at the transcription start site (TSS), transcription terminal site (TTS) or nucleosome depletion region (NDR) to predict the disease. Furthermore, the present invention constructs a prediction model based on the coverage of the nucleosome interval.

The present invention provides a disease prediction model with a relatively high accuracy and its construction method and application. The method for constructing a cell-free DNA-based disease prediction model comprises: 1) obtaining sequencing data of cell-free DNA samples of a plurality of diseased individuals and a plurality of control individuals; 2) selecting a gene set having differences in the coverage at the transcription start site regions between the diseased individuals and the control individuals according to the coverage of the sequencing data of the cell-free DNA samples of the diseased individuals and the control individuals on the genome; and 3) for the genes in the gene set, training a prediction model by inputting the coverage of the sequencing data at the gene transcription start site regions to construct a disease prediction model. The cell-free DNA-based disease prediction method comprises: 1) for the cell-free DNA sample of the individual to be tested, obtaining sequencing data of the gene set determined in constructing the disease prediction model; 2) for the genes in the gene set, obtaining the coverage of the sequencing data at the transcription start site regions; and 3) inputting the coverage at the transcription start site regions into the disease prediction model to predict whether the individual to be tested suffers from the disease. In the above two methods, the gene set used corresponds to the method for calculating the coverage of the sequencing data at the transcription start site regions.

The application of the disease prediction model includes the cell-free DNA-based disease prediction. The present invention provides a cell-free DNA-based disease prediction system, which can be used to implement the cell-free DNA-based disease prediction.

According to a specific example of the present invention, plasma cfDNA sequencing data of normal controls and patients with early lung cancer are used as input data, and the specific steps are as follows:
1. Preliminary data processing.

After the completion of quality control of all raw off-machine sequencing data (fq format) of samples used for model training, prediction and validation, reads of the sequencing data are aligned to the human reference chromosomes by using alignment software (such as samse mode in BWA); SAMtools is used to calculate the duplication rate of duplicated reads, alignment rate and mismatch rate in the alignment results, and the reads aligned to the human reference chromosomes are selected.
2. Calculation of the relative coverage value of sequencing coverage at the transcription start site region in single sample.

For each sample, sequencing depth near the transcription start site (TSS) region (the region of 100 bp, 400 bp, 600 bp, or 1 kb upstream and downstream of the transcription start site can all be used as the region near the transcription start site) is calculated for each gene in the whole genome. Different computational methods are used for single-strand and double-strand sequencing. There are two cases, including forward alignment and reverse alignment, for single-strand sequencing. In the forward alignment, the start site of alignment in the bam file is directly recorded, and in the reverse alignment, the end site of alignment in the bam file is recorded as the start site of alignment. Then, depending on the direction of alignment, backward extension is performed in the forward alignment and forward extension is performed in the reverse alignment, extending 167 bp from the start site of sequencing to the peak length of cfDNA. For the double-strand sequencing, the fragments with read 1 and read 2 just aligned to the same chromosome and with an inserted fragment length of 120 bp to 300 bp are calculated.

The average sequencing depth near the transcription start site region of each gene is calculated after locating the distribution position of fragments on the genome according to the alignment file. In order to enhance the relevant signals, only the sequencing depth of the central 61 bp of the sequencing fragment is counted, and normalization is carried out according to the overall aligned read count, to remove the differences caused by different aligned read counts and obtain the relative coverage (RC).
3. Selection of lung cancer-related genes.

For the region near the transcription start site of each gene (or transcript), the relative coverage values at the transcription start site regions of this gene of samples with lung cancer and control samples are tested for significance (general statistical monitoring methods such as rank sum test or T test can be used), and m (10-50, an appropriate value set according to the number of training samples) significantly different genes are selected as lung cancer-related genes for the subsequent construction of prediction model.
4. Construction of input matrix based on the relative coverage data at the transcription start site regions.

A prediction model is constructed by inputting the lung cancer-related gene matrix formed by the relative coverage at the transcription start site regions of the significantly different genes obtained in Step 3 corresponding to n samples used for model training. That is, the relative coverage at the region of 100 bp, 400 bp, 600 bp or 1 kb upstream and downstream of the transcription start sites of m significantly different genes corresponding to n samples is calculated to obtain the relative coverage matrix of n×m, which is used as training set D.
5. Construction of lung cancer prediction model:

Statistical software such as R can be used to conduct the training of Logistics Regression, Random Forest or other prediction model, and the final results are stored as a prediction model for the prediction of the last step.

In one embodiment, the present invention uses a model based on Random Forest (default parameters).
6. Using the constructed model to predict lung cancer.

For the sample set to be predicted, the relative coverage values at the transcription start site regions of genes obtained in Step corresponding to each sample are calculated. The m relative coverage values of each sample are taken as input and the prediction model obtained in Step 4 is used to predict whether the sample is a tumor sample.

### Example 1: Example of Application in Lung Cancer

1. Samples: The overall sample set includes 57 healthy individuals and 100 individuals with lung adenocarcinoma, as shown in Table 1.

TABLE 1
Summary of training set and test set samples
used for lung cancer prediction

Stage

| Type | Number | I | II | III | IV |
|---|---|---|---|---|---|
| Healthy | 57 | — | — | — | — |
| (Negative Samples) | | | | | |
| Lung Adenocarcinoma | 100 | 78 | 8 | 10 | 4 |
| (Positive Samples) | | | | | |

Sampling and sequencing: plasma samples of healthy individuals and patients with lung cancer were taken to extract cell-free DNA. After the experimental library was constructed, sequencing was performed using BGIseq500 with PE100 and 3× sequencing protocol.
2. Samples segmentation: training samples (N=126) and test samples (N=31) were generated by segmenting the total samples in Step 1 at a ratio of 8:2. During the process of segmentation, the ratio of positive and negative samples in training samples and test samples remained constant as that in the raw data set.
3. Selection of the genes with differential coverage at the transcription start site regions: the relative coverage values near the transcription start site regions of all genes of healthy samples and samples with lung adenocarcinoma in the training data set were calculated. Wilcox rank sum test was performed on the relative coverage values of healthy samples and samples with lung adenocarcinoma, which was implemented by wilcox test package of R statistical software in this example. Finally, genes with significant differences were selected from all genes as the features for subsequent model training. Considering the number of samples in the sample set, the top 30 genes with the lowest P-value (Table 2) were selected from all genes and defined as the genes with significant differences (the number could be less than or equal to 3×\sqrt{the number of samples}). Finally, a total of 30 genes with significant differences in the distribution of relative coverage at the regions near different transcription start sites (here, 1000 bp upstream and downstream of the transcription start site was selected as the region near transcription start site) in healthy samples and samples with lung adenocarcinoma were obtained. The relative coverage values near the transcription start sites of the 30 genes with significant differences in the training samples were extracted to generate the training set. The relative coverage values near the transcription start sites of the 30 genes with significant differences in the test samples were extracted to generate the test set.

TABLE 2
The list of screened 30 genes

Gene name: transcript ID: chromosome: position
MIR3648-2: NR_128711: chr21: 9825831
COX4I2: NM_032609: chr20: 30225690
SNX16: NM_001348189: chr8: 82754521
YBX3P1: NR_027011: chr16: 31580845
MIR3687-2: NR_128714: chr21: 9826202
GRIN2A: NM_001134407: chr16: 10276263
KLHL11: NM_018143: chr17: 40021684
HTRA1: NM_002775: chr10: 124221040
DLX4: NM_001934: chr17: 48050129
GAL3ST3: NM_033036: chr11: 65816651
MTCH1: NM_001271641: chr6: 36954327
GRIN2A: NM_001134408: chr16: 10275924
LOC101929748: NR_136301: chr9: 95570369
PMEPA1: NM_001255976: chr20: 56265680
SNORD157: NR_145781: chr19: 55914025
LOC100128531: NR_038941: chr22: 25508659
CYP4F22: NM_173483: chr19: 15619335
MIR1229: NR_031598: chr5: 179225346
DPEP3: NM_001129758: chr16: 68014452
PTGES: NM_004878: chr9: 132515344
LOC100130587: NR_110634: chr20: 61991339
MIR1250: NR_031652: chr17: 79107108
KCTD1: NM_001142730: chr18: 24128500
HNRNPAB: NM_004499: chr5: 177631507
MAFF: NM_001161572: chr22: 38597938
GUSBP4: NR_132999: chr6: 58263207
APBA2: NM_001130414: chr15: 29213839
SSBP4: NM_001009998: chr19: 18530145
LOC101927472: NR_120622: chr10: 106083121
EVA1C: NM_001320744: chr21: 33784688

4. Lung cancer prediction model
5-fold cross-validation was performed on the training set to complete the feature selection. The process was as follows:
(a) 126 samples in the training set were randomly segmented into 5 equal parts according to the ratio of positive and negative samples, wherein 4 equal parts constituted the training set and the remaining one was used as the validation set. The process was repeated 5 times to generate a 5-fold cross-validation set.
(b) Feature selection: for each training set in the above step, a Random Forest model was established and the importance of each gene in the model was output, and 10 genes with the highest importance corresponding to each model were selected. This process was repeated 5 times, and the list of important genes selected each time was shown in Table 3.

TABLE 3
List of genes selected in each round of 5-fold cross-validation.

| Rounds of 5-fold | |
|---|---|
| cross-validation | Feature gene list |
| Round 1 | HTRA1: NM_002775: chr10: 124221040 |
| | LOC101929748: NR_136301: chr9: 95570369 |
| | PMEPA1: NM_001255976: chr20: 56265680 |
| | MIR3648-2: NR_128711: chr21: 9825831 |
| | MIR3687-2: NR_128714: chr21: 9826202 |
| | PTGES: NM_004878: chr9: 132515344 |
| | DLX4: NM_001934: chr17: 48050129 |
| | MIR1250: NR_031652: chr17: 79107108 |
| | LOC101927472: NR_120622: chr10: 106083121 |
| | CYP4F22: NM_173483: chr19: 15619335 |
| Round 2 | HNRNPAB: NM_004499: chr5: 177631507 |
| | KCTD1: NM_001142730: chr18: 24128500 |
| | SNX16: NM_001348189: chr8: 82754521 |
| | MIR3687-2: NR_128714: chr21: 9826202 |
| | LOC101929748: NR_136301: chr9: 95570369 |
| | LOC100128531: NR_038941: chr22: 25508659 |
| | DPEP3: NM_001129758: chr16: 68014452 |
| | MIR3648-2: NR_128711: chr21: 9825831 |
| | MIR1229: NR_031598: chr5: 179225346 |
| | GAL3ST3: NM_033036: chr11: 65816651 |
| Round 3 | APBA2: NM_001130414: chr15: 29213839 |
| | CYP4F22: NM_173483: chr19: 15619335 |
| | HTRA1: NM_002775: chr10: 124221040 |
| | HNRNPAB: NM_004499: chr5: 177631507 |
| | YBX3P1: NR_027011: chr16: 31580845 |
| | MTCH1: NM_001271641: chr6: 36954327 |
| | GAL3ST3: NM_033036: chr11: 65816651 |
| | COX4I2: NM_032609: chr20: 30225690 |
| | PTGES: NM_004878: chr9: 132515344 |
| | DPEP3: NM_001129758: chr16: 68014452 |
| Round 4 | MIR1229: NR_031598: chr5: 179225346 |
| | GAL3ST3: NM_033036: chr11: 65816651 |
| | MTCH1: NM_001271641: chr6: 36954327 |
| | SNX16: NM_001348189: chr8: 82754521 |
| | MIR3648-2: NR_128711: chr21: 9825831 |
| | LOC101927472: NR_120622: chr10: 106083121 |
| | YBX3P1: NR_027011: chr16: 31580845 |
| | MIR1250: NR_031652: chr17: 79107108 |
| | LOC100128531: NR_038941: chr22: 25508659 |
| | LOC100130587: NR_110634: chr20: 61991339 |
| Round 5 | LOC100130587: NR_110634: chr20: 61991339 |
| | SSBP4: NM_001009998: chr19: 18530145 |
| | LOC100128531: NR_038941: chr22: 25508659 |
| | GRIN2A: NM_001134408: chr16: 10275924 |
| | CYP4F22: NM_173483: chr19: 15619335 |
| | SNORD157: NR_145781: chr19: 55914025 |
| | KLHL11: NM_018143: chr17: 40021684 |
| | MIR3648-2: NR_128711: chr21: 9825831 |
| | MIR3687-2: NR_128714: chr21: 9826202 |
| | MAFF: NM_001161572: chr22: 38597938 |

(c) The features selected by the model in each result in the above step were recorded. 5 features with the most votes were selected from all features selected from 5 cross-validations by using the majority voting rule, as shown in Table 4:

TABLE 4
List of 5 feature obtained from feature selection

Gene name: transcript ID: chromosome: position
MIR3648-2: NR_128711: chr21: 9825831
MIR3687-2: NR_128714: chr21: 9826202
LOC100128531: NR_038941: chr22: 25508659
GAL3ST3: NM_033036: chr11: 65816651
CYP4F22: NM_173483: chr19: 15619335

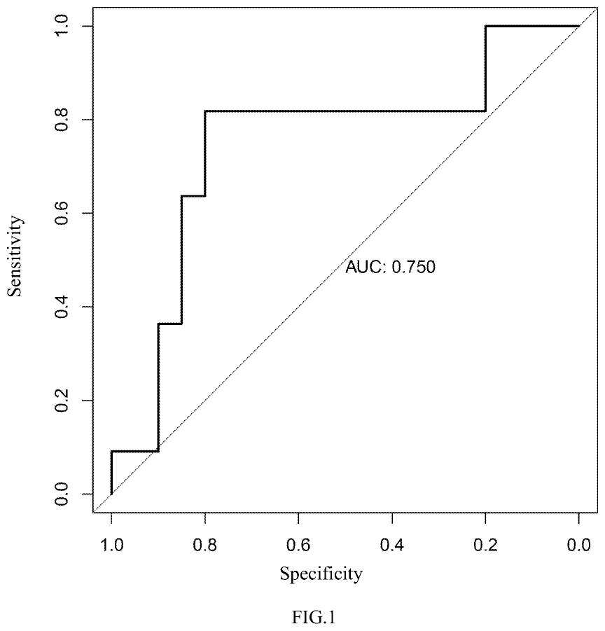

(d) Construction of final model: the feature lists in Table 4 were used to rebuild a Random Forest model.
(e) Model evaluation: evaluation of the model was performed with 31 samples in the test set. The evaluation result is shown in FIG. 1. According to FIG. 1, in the ROC curve of the test data set, the area under the curve (AUC) value can reach 0.75. In addition, according to the results of the confusion matrix of the test data set in Table 5, sensitivity and specificity can reach 0.8 and 0.73, respectively, with a precision of 0.84.

TABLE 5
Confusion matrix of the test data set

| | Predicted to be lung | |
|---|---|---|
| | adenocarcinoma | Predicted to be healthy |
| Lung | 16 | 4 |
| Adenocarcinoma | | |
| Healthy | 3 | 8 |
| Sensitivity | 0.8 | 95% Confidence Interval |
| | | (0.55, 0.93) |
| Specificity | 0.73 | 95% Confidence Interval |
| | | (0.6, 0.96) |
| Precision | 0.84 | 95% Confidence Interval |
| | | (0.39, 0.94) |

The present invention realizes lung cancer prediction with relatively high accuracy only by using the distribution of genome sequencing depth of cfDNA data in plasma obtained from one sampling, providing a concise, efficient and low-cost reference assistant means for the clinical diagnosis of lung cancer. The present invention integrates the coverage at transcription start site regions of different genes into a Random Forest model to realize the efficient early prediction of lung cancer with relatively high accuracy, and provides a comprehensive and systematic method for predicting lung cancer by using cfDNA data.

### Example 2: Example of Application in Liver Cancer

The data are derived from www.ebi.ac.uk (accession no. EGAS00001001024), sequenced by the Illumina platform with a length of pair-end reads of 75 bp, a read count in each sample of 17-79 MB, and a median of 31 MB. Please refer to Peiyong Jiang, et al. PNAS 2015 for detailed data description.

90 cell-free nucleic acid samples of liver cancer and 32 free nucleic acid samples of healthy control were included. The data were divided into the training set of 97 samples and the test set of 25 samples in the ratio of 8:2, where the ratio of samples of liver cancer to healthy samples was kept constant.

The three-step process, including preliminary data processing, calculation of the relative coverage value of sequencing coverage at the transcription start site region in single sample, and selection of liver cancer-related genes, was same to the previous description. After the Wilcox rank sum test was performed according to the relative coverage near the transcription start sites between two groups, 25 differential genes were screened as features by P value from small to large in the training set. A Random Forest model was built based on the training data set and then was applied to the test data set. The results are shown as follows:

TABLE 6
Screened 25 gene lists as the final classification features

Gene name: transcript ID: chromosome: position
MIR514A3: NR_030240: chrX: 146363548
NBEAP1: NR_027992: chr15: 20961480
MIR4477A: NR_039688: chr9: 68415388
MIR4477B: NR_039689: chr9: 68415307
PDE4DIP: NM_001350521: chr1: 145076186
LINC01262: NR_121679: chr4: 190580759
MIR3687-2: NR_128714: chr21: 9826202
PDE4DIP: NM_001198832: chr1: 145039995
DRD5P2: NR_111001: chr1: 148901844
LOC101060524: NR_111000: chr1: 148901844
MIR3648-2: NR_128711: chr21: 9825831
LOC100996724: NR_144516: chr1: 145039963
LOC101927237: NR_110747: chr4: 68287718
PTGER4P2-CDK2AP2P2: NR_135010: chr9: 66496468
MIR8078: NR_107045: chr18: 112339
SRGAP2-AS1: NR_104189: chr1: 121139765
USP17L18: NM_001256859: chr4: 9250355
LOC440570: NR_135765: chr1: 17197439
PTGER4P2-CDK2AP2P2: NR_024496: chr9: 66494268
MIR663B: NR_031608: chr2: 133014653
ANKRD30BL: NR_152415: chr2: 133015542
LINC01660: NR_136569: chr22: 20656828
ZNF806: NM_001304449: chr2: 133064716
LOC101927050: NR_136329: chr2: 91900136
SEC22B: NM_004892: chr1: 145096406

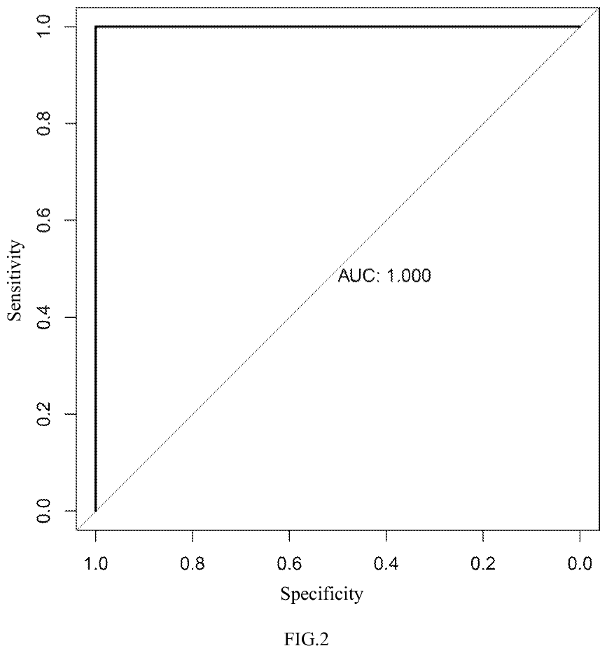

The ROC curve of the test set is shown in FIG. 2. In addition, according to the confusion matrix result of the test data set (see Table 7), sensitivity, specificity and accuracy of this method can reach 1 in the liver cancer prediction.

TABLE 7
Confusion matrix results

| | Predicted to be | |
|---|---|---|
| | liver cancer | Predicted to be healthy |
| Liver Cancer | 19 | 0 |
| Healthy | 0 | 6 |
| Sensitivity | 1 | 95% Confidence Interval (0.79, 1) |
| Specificity | 1 | 95% Confidence Interval (0.52, 1) |
| Precision | 1 | 95% Confidence Interval (0.79, 1) |

1. A method for constructing a cell-free DNA-based disease prediction model, comprising:
1) obtaining sequencing data of cell-free DNA samples of a plurality of diseased individuals and a plurality of control individuals;
2) selecting a gene set having differences in the coverage at the transcription start site regions between the diseased individuals and the control individuals according to the coverage of the sequencing data of the cell-free DNA samples of the diseased individuals and the control individuals on the genome; and
3) for the genes in the gene set, training a prediction model by inputting the coverage of the sequencing data at the gene transcription start site regions to construct a disease prediction model.

2. The method of claim 1, wherein the disease is cancer, and the disease prediction includes early screening of tumors or detection of tumor recurrence.

3. The method of claim 1, in 1), wherein the cell-free DNA samples are derived from body fluids.

4. The method of claim 1, wherein the coverage of the cell-free DNA on the genome is determined by the relative coverage.

5. The method of claim 1, wherein the transcription start site region refers to a region of 100 bp, 400 bp, 600 bp, or 1 kb upstream and downstream of the transcription start site.

6. The method of claim 1, wherein the gene set comprises 10-50 genes.

7. The method of claim 1, wherein the prediction model is a Logistic Regression model or a Random Forest model.

8. (canceled)

9. A cell-free DNA-based disease prediction method, which uses the disease prediction model constructed by the method of claim 1, comprising:
1) for the cell-free DNA sample of an individual to be tested, obtaining sequencing data of the gene set determined in constructing the disease prediction model;
2) for the genes in the gene set, obtaining the coverage of the sequencing data at the transcription start site regions; and
3) inputting the coverage at the transcription start site regions into the disease prediction model to predict whether the individual to be tested suffers from the disease.

10. (canceled)

11. The method of claim 2, wherein the cancer is lung cancer, liver cancer, or colorectal cancer.

12. The method of claim 3, wherein the body fluid is blood.
